(19) United States
(12) Patent Application Publication
(10) Pub. No.: US 2007/0202577 A1
(43) Pub. Date: Aug. 30, 2007

(54) Method For The Production Of Hyperbranched Polysaccharide Fractions

(76) Inventor: Klaus Sommermeyer, Rosbach v.d.H. (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/590,676

(22) PCT Filed: Feb. 26, 2005
(86) PCT No.: PCT/EP05/02057

§ 371 Date: Nov. 28, 2006

(30) Foreign Application Priority Data
Feb. 28, 2004 (DE) 10 2004 009 783.6

(51) Int. Cl. C12P 19/04; C08B 31/00

(52) U.S. Cl. 435/101; 536/45

(57) ABSTRACT

The invention relates to a method for producing hyperbranched amylopectin having a mean molecular weight ranging between 2,000 and 29,000 Dalton and an average degree of branching of more than 10 percent and less than 20 percent, said degree of branching being expressed in mole percent of the anhydroglucose units carrying branching points. According to the inventive method, the molecular weight of plant amylopectins or starch rich in amylopectin is reduced to molecular weights not exceeding 60,000 Dalton by means of a-amylase or acid hydrolysis in a first hydrolysis step, and the molecular weight of the reduced product obtained in the first hydrolysis step is further reduced by means of β-amylase reduction in a second hydrolysis step. The invention further relates to the production of coupling products of the hyperbranched amylopectin with a pharmaceutical agent.

The present invention relates to a method for the production of hyperbranched amylopectin and a method for the production of products of the coupling of a hyperbranched amylopectin with active pharmaceutical ingredients.

It has emerged that the side effects of active pharmaceutical ingredients which are administered parenterally can be reduced by coupling hydrophilic polymers thereto. It is possible in particular by increasing the molecular weight of these active ingredients to reduce or even prevent renal side effects if the molecular size of the products of the coupling is above the exclusion limit of the kidney, which acts like a filter. The molecular size of the product of the coupling is in this connection adjusted through the appropriately selected molecular weight of the polymer.

A further advantage of a product of the coupling of hydrophilic polymer and active pharmaceutical ingredient is that the antigenicity of therapeutic proteins is reduced, and thus the side effects relating thereto can be reduced or prevented.

It is likewise possible to extend considerably the pharmacokinetic half lives and thus the residence times of the active pharmaceutical ingredients in the patient's serum through such products of coupling. This makes it possible to extend considerably the therapy intervals on parenteral administration.

Polymers suitable for the coupling to active pharmaceutical ingredients described above are in particular polyethylene glycols [Herman, S. et al., Poly(Ethylene Glycol) with Reactive Endgroups: I. Modification of Proteins, Journal of Bioactive and Compatible Polymers, 10. (1995) 145-187] or else polysaccharides, for example starch derivatives and dextrans. Appropriate activation is followed by coupling to the active ingredients.

The active ingredients are in this case coupled to the carrier molecules by chemical methods which are known per se and which are already known from the technique of immobilizing ligands on solid phases or from the chemistry of protein coupling or crosslinking. Appropriate methods are described in G. T. Hermanson et al., Immobilized Affinity Ligand Techniques, Academic Press Inc. (1992) and in S. S. Wong, Chemistry of Protein Conjugation and Cross-Linking, CRC Press LLC (1993) and C. P. Stowell et al., Neoglycoproteins, the preparation and application of synthetic Glycoprotein, In: Advances in Carbohydrate Chemistry and Biochemistry, Vol. 37 (1980), 225-281.

Disadvantages of polyethylene glycols compared with starch derivatives in this connection is that they cannot be directly metabolized in the body, whereas the starch derivatives can be degraded by endogenous serum α-amylase. Degradation of the starch derivatives in the body can be deliberately delayed by suitable substitution, e.g. with hydroxyethyl groups, making it possible to tailor the kinetics of the active ingredient conjugates which can be administered parenterally [K. Sommermeyer et al., Krankenhauspharrnazie, volume 8, no. 8, (1987)].

However, a disadvantage of the derivatization of starch with hydroxy groups is that, owing to the preparation, the distribution of the hydroxyethyl groups along the chain is non-uniform, and thus, owing to the regionally high degrees of substitution at certain points in the carbohydrate chain, fragments which cannot be further degraded by endogenous enzymes are formed during degradation in the body. These fractions form the so-called storage fractions [P. Lawin, et al., Hydroxyethylstarke, Eine aktuelle Übersicht, Georg Thieme Cerlag (1989)].

DE 102 17 994 describes hyperbranched polysaccharides for coupling to active pharmaceutical ingredients. These disclosed hyperbranched amylopectins have a structure similar to that of endogenous glycogen and are therefore extremely well tolerated and completely degradable in the body. It is possible by adjusting the degrees of branching to adjust the kinetics of degradation of the hyperbranched amylopectins in such a way that the desired residence times in the serum can be achieved without further derivatization.

Concerning the production of these hyperbranched amylopectins, DE 102 17 994 refers to EP 1 369 432. EP 1 369 432 discloses soluble, hyperbranched glucose polymers with a proportion of α-1,6-glycosidic linkages of >10%, preferably between 12 and 30%, and a molecular weight of between 35 000 and 200 000 daltons. According to EP 1 369 432, these polymers are produced by treating an aqueous suspension of starch or solution of starch with a branching enzyme in order to increase the degree of branching, and subsequently hydrolyzing with an enzyme selected from the group of α-amylase, β-amylase, anhydroglycosidase and α-transglucosidase. The branching enzyme required for this purpose is extracted from organisms and/or microorganisms and is selected from the group consisting of glycogen branching enzymes, starch branching enzymes and mixtures of these enzymes

A disadvantage of the method described in EP 1 369 432 is that it is elaborate and costly. Especially the use of branching enzymes, which are not at present commercially available, means that extra isolation thereof is necessary in each case from organisms and/or microorganisms.

It is thus objects of the invention to provide a simple and cost-effective method for producing hyperbranched polysaccharides which can be used as carrier molecules for active pharmaceutical ingredients.

It has surprisingly been found that a method as claimed in claim 1 achieves this object. This entails in a first hydrolysis step degrading vegetable amylopectins or amylopectin-rich starches by α-amylase or acid hydrolysis to molecular weights of less than or equal to 60 000 daltons, and a second hydrolysis step further degrading the molecular weight of the degradation product from the first step by a β-amylase degradation.

It has further been found that it was possible to obtain a marked increase in the degree of branching by the acid hydrolysis of amylopectin or amylopectin-rich starches to weight-average molecular weights of less than or equal to 60 000.

Such a hyperbranched amylopectin corresponding to the present invention preferably has a weight-average molecular weight of ≧2000 daltons and a degree of branching of ≧10%. A weight average molecular weight of ≧2000 daltons and ≦29 000 daltons and a degree of branching of ≧10% and ≦20% is particularly preferred.

Amylopectins mean in this connection in the first place very generally branched starches or starch products with α-(1-4) and α-(1-6) linkages between the anhydroglucose units. The branches in the chains come about in this case through the α-(1-6) linkages. These branch points are present irregularly about every 15 to 30 glucose elements in naturally occurring amylopectins. The molecular weight of natural amylopectin is very high in the range from 10⁷ to 2×10⁸ daltons. It is assumed that amylopectin also forms helices within certain limits.

A degree of branching can be defined for amylopectins. The measure of the branching is the ratio of the number of anhydroglucose units which have branch points [α-(1-6) linkages] to the total number of anhydrogluclose units in the amylopectin. This ratio is expressed in mol %. Amylopectin occurring in nature has degrees of branching of about 4 mol %. Hyperbranched amylopectins have a degrees of branching which are markedly increased compared with the degrees of branching occurring in nature. The degree of branching in this connection is in every case an average (average degree of branching) because amylopectins are polydisperse substances.

In the context of this invention, hyperbranched amylopectins are intended to mean amylopectins with an average degree of branching of greater than or equal to 10 mol %.

Degradation of vegetable amylopectins or amylopectin-rich starches with α-amylase or acid hydrolysis results, depending on the respective degree of hydrolysis of the hydrolysis products, in amylopectins with a similar degree of branching in each case. In this connection, degradation by acid hydrolysis is easier to carry out and cheaper than enzymatic degradation with α-amylase. It is further possible with acid hydrolysis to follow the degree of hydrolysis during the hydrolysis process by in-process HPGPC and to adjust the degree of hydrolysis deliberately. Degradation by acid hydrolysis is thus particularly preferred over degradation with α-amylase.

β-Amylase treatment of the products obtained in the first hydrolysis step degrades them selectively on the α-1,4-glycosidic anhydroglucose units. In this degradation there is elimination of the maltose units at the outer, non-reducing chain ends, without the α-1,6-glycosidic branches themselves being disconnected. Degradation in this case takes place from the outer chain end as far as about 2 glucose units in front of the first occurring branch point. This results in the so-called β-genzdextrins in which the 1,6-glycosidic linkages of the amylopectin are enriched and thus the degree of branching is increased.

In the context of the present invention, all amylopectin-containing starches can be used as starting material. Waxy corn starch and cassava starch are particularly preferred in this connection.

Owing to the high degree of branching, the β-genzdextrins are correspondingly slowly degraded in serum because α-amylase predominates there for degrading polysaccharides. The products from the method of the invention are therefore suitable for coupling to active pharmaceutical ingredients.

The parameters of degree of branching and molecular weight of the amylopectin allow targeted influencing and thus adjustment of desired pharmacokinetics, in particular attainment of a desired α-amylase degradation. The degree of branching of the amylopectin has a key function in this connection, both the molecular weight also has an influence on the kinetics mentioned. It is moreover possible to influence the kinetics of degradation of amylopectin in a desired direction also through the distribution of the branching products.

In the method of the invention preferably low molecular weight impurities with an absolute molecular weight of <5000 daltons, preferably <1000, are removed after the first hydrolysis step and/or after the second hydrolysis step. This removal preferably takes place by ultrafiltration, using membranes having a cutoff of 5000 daltons or 1000 daltons. The removed impurities are mainly low molecular weight degradation products of amylopectin and of starch, and hydrochloric acid.

The product degraded according to the invention is preferably isolated by freeze drying.

α- and β-amylase are commercially available, cost-effective enzymes. Hydrolysis with these molecules can therefore be carried out simply and cost-effectively. The same applies to acid hydrolysis. The working up by ultrafiltration and freeze drying is also simple and not costly. The products of the invention can therefore be produced simply and cost-effectively.

The hydrolysis product of the second hydrolysis step is preferably coupled to an active pharmaceutical ingredient. The active pharmaceutical ingredient is preferably a protein or a polypeptide.

The coupling of the hyperbranched amylopectin produced according to the invention to the active pharmaceutical ingredient can take place in a known manner. Such couplings of an active pharmaceutical ingredient to a polysaccharide are described for example in WO 02/08 0979, PCT/EP 02/06 764, WO 03/07 4088, WO 03/07 4087, PCT/EP 03/13 622, DE 102 54 754.9 and PCT/EP 04/00 488.

The active pharmaceutical ingredient is preferably coupled via a free amino function to the anhydroglucose units of the reducing chain end of the hyperbranched amylopectin. For this purpose, the reducing end of the hyperbranched amylopectin is particularly preferably activated. It is particularly preferred in this connection to oxidize the reducing ends of the hyperbranched amylopectin to the aldonic acid, to activate the aldonic acid group to the aldonic acid ester group, and to couple the active pharmaceutical ingredient to the hyperbranched amylopectin via the aldonic acid ester group. It is likewise preferred to react the product produced according to the invention in anhydrous medium with a carbonic acid diester to give a carbonic acid diester of the hyperbranched amylopectin and to couple the latter to the active ingredient.

The invention is explained in more detail below by means of examples and comparative examples, without intending to restrict the invention to these examples.

Measurement Methods

The molecular weight and the weight average molecular weight were determined by conventional methods. These include for example aqueous GPC, HPGPC, HPLC, light scattering and the like.

The degree of branching was determined by means of ¹H NMR.

### EXAMPLE 1

55 g of thin-boiling waxy corn starch were suspended in 1000 ml of deionized water, and the suspension was brought to boiling under reflux. The waxy corn starch was completely dissolved thereby. After dissolving, the pH was adjusted to a pH of 2.0 with 1N HCl, and the mixture was heated under reflux for one hour. After cooling, ultrafiltration was carried out with a membrane with a nominal cutoff of 5000 daltons against deionized water. The substance purified in this way was isolated by freeze drying. The yield was 60%. Characterization of the substance revealed a weight average molecular weight of 42 000 daltons (measured by HPGPC) and a degree of branching of 7 mol % (measured by ¹H NMR).

### EXAMPLE 2

10 g of the waxy maize starch degraded fraction from example 1 were dissolved in 1000 ml of 0.15 molar acetate buffer, pH 4.2, and 10 units/ml β-amylase (from Sigma, β-amylase type I-B from sweet potato, Art. No. A7005) were added. The mixture was allowed to react at 25° C. for 12 hours. The enzyme was then inactivated by boiling the mixture at 100° C. for 10 minutes. After cooling, about 2% by weight of activated carbon (based on the substrate) were added to the reaction mixture and filtered off. Subsequently, the maltose and the buffer were removed by ultrafiltration of the reaction product using a membrane with a cutoff of 1000 daltons, and the β-genzdextrin was isolated by freeze drying. The yield was 60%. Characterization revealed a degree of branching of 14 mol % (measured by ¹H NMR) and a weight average molecular weight of 28 000 daltons.

### EXAMPLE 3

Example 3 was carried out in analogy to example 1, prolonging the hydrolysis time to 4 hours. In this case, the hydrolysis method was followed by in-process HPGPC in order to obtain a product with a weight average molecular weight of <15 000 daltons. Purification by ultrafiltration followed in contrast to example 1 with the aid of a membrane having a nominal cutoff of 1000 daltons. The yield was 25%. Characterization of the substance revealed a weight average molecular weight of 10 000 daltons and a degree of branching of 10.3 mol %.

### EXAMPLE 4

The β-genzdextrin was produced in analogy to example 2, using the hydrolysis product from example 3. The yield was 60%. Characterization of the substance revealed a weight average molecular weight of 7000 daltons and a degree of branching of 15 mol %.

### EXAMPLE 5

55 g of native cassava starch were gelatinized in 1000 ml of deionized water heating under reflux. Then 11 ml of 1N HCl were added to adjust a pH of about 1.9. After 30 minutes, the gel was of low viscosity and the mixture was heated under reflux for a further 7 hours. After cooling, the precipitate and the turbidity were filtered off, and ultrafiltration was carried out against deionized water with a membrane with a nominal cutoff of 1000 daltons. The yield was 24.4%. Characterization of the substance revealed a weight average molecular weight of 10 000 daltons and a degree of branching of 9.6 mol %.

### EXAMPLE 6

The β-genzdextrin was produced in analogy to example 2, with the difference that the hydrolysis substance from example 5 was employed. The yield was 55%. Characterization of the substance revealed a weight average molecular weight of 5000 daltons and a degree of branching of 16 mol %.

### EXAMPLE 7

The waxy corn starch degradation fraction from example 2 was dissolved in isotonic phosphate buffer of pH 7.2 to result in a 1% by weight solution. The solution was heated to 37.0° C., and 0.5 I.U./ml α-amylase from porcine pancreas (from Roche; AS, Art. No. 102 814) was added. Samples were taken after 1 and 3 hours, the enzyme was inactivated by heat, and the molecular weight of the remaining high molecular weight fraction was determined by HPGPC. In this case, the initial weight average molecular weight, was 28000 daltons, the weight average molecular weight after hydrolysis for 1 hour was 11 000 daltons and the weight average molecular weight after hydrolysis for 3 hours was 7000 daltons.

### EXAMPLE 8

The method of example 7 was repeated employing the degradation fraction from example 4. In this case, the initial weight average molecular weight was 7000 daltons, the weight average molecular weight after hydrolysis for 1 hour was 5500 daltons and the weight average molecular weight after hydrolysis for 3 hours was 4600 daltons.

### Comparative Experiment 1

Comparative experiment 1 was carried out in analogy to example 7 employing commercially available hydroxyethyl starch (130/0.4, proprietary name “Voluven”) instead of the degradation fraction from example 2. The initial weight average molecular weight was 140 200 daltons, the weight average molecular weight after 1 hour was 54 700 daltons. The weight average molecular weight after hydrolysis for 3 hours was 33 700 daltons.

The rate of degradation of the commercially available plasma expander based on hydroxyethylstarch with α-amylase from comparative experiment 1 is thus comparable to the rate of degradation of the hyperbranched amylopectin fraction from example 7.

### EXAMPLE 9

Oxidation of the hyperbranched amylopectin fraction from example 4 at the reducing end group to the aldonic acid.

A 25% by weight solution in deionized water of the hyperbranched degradation fraction produced in example 4 was prepared. A 3.5-fold molar excess, based on the reducing end group, of a 0.05 molar iodine solution was slowly added in portions to this solution and was removed in portions in each case with 0.1N NaOH (3 times the molar quantity based on iodine). After the addition, reaction was allowed to continue at room temperature overnight, and the resulting solution was then dialyzed with a membrane with a nominal cutoff of 1000 daltons, monitoring the pH. After a pH in the dialysate of about 6 was reached and freedom from iodide had been checked by adding sodium iodate and acidifying, the mixture was adjusted to pH 2.5 with 0.1N HCl and dialyzed further until the ultrafiltrate had a pH of 5. The product was isolated by freeze drying. The yield was 80% of the theoretical yield. The degree of oxidation was >90% and was determined via the reducing end group.

### EXAMPLE 10

66 mg of aldonic acid from example 9 were dissolved in 0.5 ml of dry DMF, and 3.4 mg of N,N′-disuccinimidyl carbonate were added and allowed to react at room temperature for 2 hours. 0.5 ml of a 1% by weight solution of bovine serum albumin (BSA) was mixed with 180 ml of a 1 molar bicarbonate solution and then two portions each of 100 μl of the activated aldonic acid were added dropwise to the BSA solution and allowed to react in each case for half an hour. The mixture was then adjusted to a pH of 7.4 with hydrochloric acid. Investigation of the reaction solution by HPGPC revealed a yield of product of the coupling of >95% of the BSA employed.

1-8. (canceled)

9. A method for the production of hyperbranched amylopectin, comprising:
(i) degrading the molecular weight of vegetable amylopectins or amylopectinrich starch by α-amylase or acid hydrolysis to molecular weights of less than or equal to 60 000 daltons; and
(ii) further degrading the molecular weight of the degradation product from step (i) by a β-amylase degradation,
wherein the product of step (ii) has an average molecular weight greater than or equal to 2000 daltons and less than or equal to 30 000, and
further wherein the product of step (ii) has an average degree of branching, expressed in mol % of the anhydroglucose units having branch points, of greater than 10% and less than or equal to 20%.

10. The method as claimed in claim 9, in which low molecular weight impurities with an absolute molecular weight of less than 5000 daltons are removed after step (i) and/or after step (ii).

11. The method as claimed in claim 9, wherein step (i) is an acid hydrolysis step.

12. The method as claimed in any of claim 9, further including the step of coupling the hydrolysis product step (ii) to an active pharmaceutical ingredient.

13. The method as claimed in claim 12, wherein the active pharmaceutical ingredient is a protein or a polypeptide.

14. The method as claimed in claim 12, wherein the coupling of the hydrolysis product of step (ii) to the active pharmaceutical ingredient takes place at the terminal anhydroglucose unit of the hydrolysis product.

15. The method as claimed in claim 14, further including the steps of
oxidizing the terminal reducing end group of the hydrolysis product of step (ii) to the aldonic acid;
activating the aldonic acid group to the aldonic acid ester group; and
coupling the active pharmaceutical ingredient to the activated aldonic acid group.

16. The method as claimed in claim 14, wherein the coupling of the hydrolysis product of step (ii) to the active pharmaceutical ingredient takes place via a carbonic acid ester group.

17. The method as claimed in claim 9, in which low molecular weight impurities with an absolute molecular weight of less than 1000 daltons are removed after step (i) and/or after step (ii).
